(19) United States
(12) Patent Application Publication
(10) Pub. No.: US 2020/0376253 A1
(43) Pub. Date: Dec. 3, 2020

(54) FUNNEL-SHAPED PRESSURIZATION VALVE

(71) Applicant: Cook Medical Technologies LLC, Bloomington, IN (US)

(72) Inventors: Ralf SPINDLER, Solsberry, IN (US); Dennis MADDOX, Spencer, IN (US); Becky BAUMGARTNER, Bloomington, IN (US)

(73) Assignee: Cook Medical Technologies LLC, Bloomington, IN (US)

(21) Appl. No.: 16/429,917

(22) Filed: Jun. 3, 2019

(51) Int. Cl. A61M 39/06

(52) U.S. Cl. CPC A61M 39/0606; A61B 2017/00867

(57) ABSTRACT

The disclosure is directed to a system for maintaining hemostasis during introducing or withdrawing an interventional device. The system includes a housing. The housing includes a first end, a second end and a side wall defining a housing chamber. The system also includes a first funnel-shaped member disposed at least partially within the housing chamber. The first funnel-shaped member includes an inner portion, a middle portion and an outer portion. The inner portion of the first funnel-shaped member includes an elastic orifice, which is aligned with the through channel of the housing. The first funnel-shaped member includes an open configuration and a closed configuration. When the first funnel-shaped member is in the closed configuration, the elastic orifice is substantially closed, and when the first funnel-shaped member is in the open configuration, an interventional device is introduced through the system and the elastic orifice is open.

### BACKGROUND

### 1. Technical Field

The present disclosure relates to a device for use in medical operation and for inserting an interventional device into a body cavity. In particular, the present disclosure relates to a hemostasis valve comprising a funnel-shaped pressurization member.

### 2. Background Information

Endovascular repair may become necessary in cases of separation of the aorta's layers (dissection), narrowing of the aorta (stenosis), and traumatic damages of the aorta (transection). For example, endovascular repair of abdominal aortic aneurysms and thoracic aortic aneurysms are typically treated by placing a stent graft inside the affected vessels. The task of stent grafts is to keep the blood vessels open, to allow sufficient blood flow, and to prevent further vessel wall expansion, thus, lowering the risk of a detrimental vessel rupture.

During clinical procedures of placing an interventional device into a body cavity, for example, placing a stent graft inside an affected vessel, one or more invention devices need to be frequently taken in and out from the body cavity. A hemostasis valve would be needed for maintaining hemostasis during the introduction or withdrawal of the interventional devices. However, the existing hemostasis valves have the disadvantages, for example, of being not reliable and leaking when using different cannula sizes.

The present disclosure is directed toward addressing one or more drawbacks, including but not limited to those set forth above.

### BRIEF SUMMARY

The present disclosure is directed to a system for maintaining hemostasis during introducing or withdrawing an interventional device. The system includes a housing. The housing includes a first end, a second end and a side wall defining a housing chamber between the first end and the second end. The housing includes a through channel disposed along a central axis of the housing. The system also includes a first funnel-shaped member disposed at least partially within the housing chamber. The first funnel-shaped member includes an inner portion, a middle portion and an outer portion. The central portion of the first funnel-shaped member includes an elastic orifice, which is aligned with the through channel of the housing. The first funnel-shaped member includes an open configuration and a closed configuration. When the first funnel-shaped member is in the closed configuration, the elastic orifice is substantially closed, and when the first funnel-shaped member is in the open configuration, an interventional device is introduced through the system and the elastic orifice is open.

### BRIEF DESCRIPTION OF THE DRAWINGS

FIG. 1A is a schematic diagram of a cross-section view of a hemostatic valve in a closed configuration.

FIG. 1B is schematic diagram of a cross-section view of a hemostatic valve in an open configuration when an interventional device is introduced.

FIG. 2A is a schematic diagram of a cross-section view of another hemostatic valve in a closed configuration.

FIG. 2B is schematic diagram of a cross-section view of another hemostatic valve in an open configuration when an interventional device is introduced.

FIG. 2C is a schematic diagram of a cross-section view of another hemostatic valve in a closed configuration.

FIG. 3A is a schematic diagram of a cross-section view of another hemostatic valve in a closed configuration.

FIG. 3B is schematic diagram of a cross-section view of another hemostatic valve in an open configuration when an interventional device is introduced.

FIG. 3C is a schematic diagram of a cross-section view of another hemostatic valve in a closed configuration.

FIG. 3D is a schematic diagram of a cross-section view of another hemostatic valve in a closed configuration.

FIGS. 4A-4D are schematic diagrams of cross-section views of hemostatic valve systems including two hemostatic valves disposed at various orientations. The hemostatic valves are in a closed configurations.

FIGS. 5A-5B are schematic diagrams of cross-section views of hemostatic valve systems including two hemostatic valves when an interventional device is introduced.

FIGS. 6A-6B are schematic diagrams of another hemostatic valve.

### DETAILED DESCRIPTION OF THE DRAWINGS

The invention will now be described in detail hereinafter with reference to the accompanied drawings, which form a part of the present invention, and which show, by way of illustration, specific examples of embodiments. Please note that the invention may, however, be embodied in a variety of different forms and, therefore, the covered or claimed subject matter is intended to be construed as not being limited to any of the embodiments to be set forth below. Please also note that the invention may be embodied as methods, devices, components, or systems. Accordingly, embodiments of the invention may, for example, take the form of hardware, software, firmware or any combination thereof.

Throughout the specification and claims, terms may have nuanced meanings suggested or implied in context beyond an explicitly stated meaning. Likewise, the phrase “in one embodiment” or “in some embodiments” as used herein does not necessarily refer to the same embodiment and the phrase “in another embodiment” or “in other embodiments” as used herein does not necessarily refer to a different embodiment. It is intended, for example, that claimed subject matter includes combinations of exemplary embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

The present disclosure relates to a hemostatic valve system, which may solve or alleviate the drawbacks as previously discussed. The present disclosure improves the reliability of sealing and may be used for different profiles of interventional devices, for example but not limited to, various sheaths and cannulas.

The hemostatic valve may consist at least of a funnel-shaped disk or balloon which is disposed inside a housing. The funnel-shaped disk or balloon may have a small hole in the center which can be expanded by an interventional device to large diameters. The hole may collapse when the introducer is removed. The seal is created by the contact area between the funnel-shaped disk or balloon and the outer surface along the interventional device. The seal may be improved by applying mechanical pressures (e.g. inside the balloon or by means of a spring mechanism). When the housing is attached to a sheath, the hemostatic valve may effectively stop blood from flowing through. One or more hemostatic valves may be disposed inside one housing to improve the sealing properties. The disk or balloon may be funnel-shaped so different sizes of the interventional device can be inserted and have similar sealing and/or friction properties.

In the present disclosure, the term “proximal end” is used when referring to that end of a medical device closest to the heart after placement in the human body of the patient, and may also be referred to as the inflow end (the end that receives fluid first), and the term “distal end” is used when referring to that end opposite the proximal end, or the one farther from the heart after its placement, and may also be referred to as the outflow end (that end from which fluid exits).

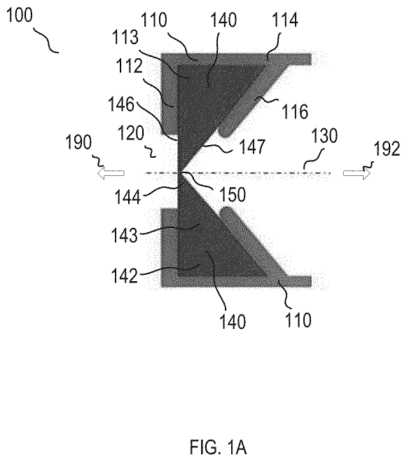

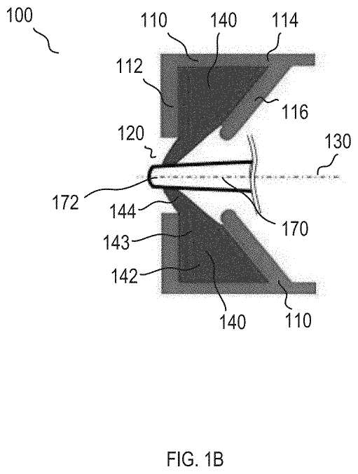

The present disclosure discloses a hemostatic valve 100 as shown in FIGS. 1A and 1B. The hemostatic valve 100 may include a housing 110 and a funnel-shaped member 140. The housing 110 may have a cylindrical outer shape with a central axis 130. The housing 110 may have a central through portion 120, which may be a through hole and have the same central axis 130. The central through portion 120 may have an inner diameter larger than a size of 24 French Gauge (Fr.), so that an interventional device with a size equal or smaller than 24 Fr. may be inserted through the central through portion 120.

The housing 110 may have an outer wall 114, a proximal member 112 disposed at a proximal end of the housing 110, a distal member 116 disposed at a distal direction 192 relative to the proximal member 112. The outer wall 114, the proximal member 112, and the distal member 116 may define a housing chamber 113. The housing chamber 113 may have a central through portion 120, which may be a through hole and have a central axis 130.

The proximal member 112 may be perpendicular or oblique to the outer wall 114. In one implementation, the proximal member 112 is perpendicular to the outer wall 114, as shown in FIG. 1A. In another implementation, the proximal member 112 may be oblique to the outer wall 114 at an angle of between 30 degrees and 90 degrees. The proximal member 112 may be oblique relative to the outer wall 114 either toward a proximal direction 190 or toward a distal direction 192.

The distal member 116 may be oblique or perpendicular to the outer wall 114. In one implementation, the distal member 116 is oblique to the outer wall 114 at about 45 degrees towards the proximal direction 190, as shown in FIG. 1A. The angle may be chosen based on shapes of commonly used introducer tips. The angle may have an influence on the insertion forces and the ability of the arrangement to guide an introducer to the center, so that a valve disk (e.g. a silicone disk) is not punctured by an introducer or guide wire. For example, the distal member 116 may be oblique to the outer wall 114 at an angle between about 30 degrees and about 90 degrees, inclusive. Here, “about” a value may mean a range of ±10% of the value. In one implementation, when an introducer includes a sharp tip, the distal member 116 may be oblique to the outer wall 114 at a smaller angle than 45 degrees, including but not limited to, 20 degrees, 30 degrees, and 40 degrees. In another implementation, when an introducer includes a blunt tip, the distal member 116 may be oblique to the outer wall 114 at a larger angle than 45 degrees, including but not limited to, 50 degrees, 60 degrees, and 70 degrees.

The hemostatic valve system may be formed from any suitable materials. The housing 110 may be made of any suitable rigid materials, for example, stainless steels, aluminum, metal alloy, and polymeric materials. The suitable polymeric materials may include, for example but not limited to, silicone, polyamide (nylon), polyurethane, polyether ether ketone (PEEK), polyester, polyethylene, polyethylene oxide, polystyrene, polypropylene, or blends or copolymers thereof.

The funnel-shaped member 140 may be disposed inside and secured by the housing 110. In one implementation as shown in FIG. 1A, the funnel-shape member 140 may be disposed inside the housing chamber 113. The funnel-shaped member 140 may include an outer portion 142, a middle portion 143, and an inner portion 144.

The outer portion 142 of the funnel-shaped member 140 may be partially enclosed by the housing 110. In one implementation as shown in FIG. 1A, the outer portion 142 of the funnel-shaped member 140 may have an outer shape similar to the inner shape of enclosure enclosed by the proximal member 112, the outer wall 114, and the distal member 116 of the housing 110, so that the funnel-shaped member 140 may be secured by the housing 110.

The inner portion 144 of the funnel-shaped member 140 may have an elastic orifice 150. The elastic orifice 150 may be at the center of the inner portion 144 and may be aligned with the central axis 130.

When no interventional device is introduced as shown in FIG. 1A, the hemostatic valve 100 may be in a closed configuration, the elastic orifice 150 is closed under an elastic force of the inner portion 144 of the funnel-shaped member 140. The closed orifice efficiently provides a seal so that substantially no liquid under a normal hydraulic pressure may pass through the elastic orifice 150. For example but not limited to, no blood under normal human blood pressure may pass through the closed orifice.

When an interventional device 170 is introduced as shown in FIG. 1B, the hemostatic valve 100 may be in an open configuration, and the elastic orifice may be expanded by the interventional device 170 to an open configuration so that a proximal end 172 of the interventional device 170 may pass through. Under the elastic force of the funnel-shaped member 140, a liquid-tight seal may be created by a contact area between the inner portion 144 of the funnel-shaped member 140 and an outer surface of the interventional device 170. The elastic orifice and the inner portion of the funnel-shaped member may bend along an outer surface of the interventional device so as to increase the contact area between the inner portion of the funnel-shaped member and the interventional device. In this configuration, when the interventional device 170 feeds further towards the proximal direction, the liquid-tight seal between the inner portion 144 of the funnel-shaped member 140 and the outer surface of the interventional device 170 may be maintained under the elastic force of the funnel-shaped member 140. For example but not limited to, the liquid-tight seal between the inner portion 144 of the funnel-shaped member 140 and the outer surface of the interventional device 170 may prevent blood under normal human blood pressure from passing through the hemostatic valve 100. When the interventional device 170 is removed, the orifice may collapse again under the elastic force of the funnel-shaped member 140 so that the hemostatic valve 100 returns to the closed configuration again. The interventional device 170 may have various sizes (for example, from 8 Fr. to 24 Fr. inclusive), and may have various profiles, for example but not limited to, a circular profile, an oval profile, a triangle profile, a square profile, a hexagon profile, etc.

The funnel-shaped member 140 may have a proximal end 146 and a distal end 147. The proximal end 146 of the funnel-shaped member 140 faces towards the proximal direction 190. The distal end 147 of the funnel-shaped member 140 faces towards the distal direction 192. In one implementation, the distal end 147 includes a profile as shown in FIG. 1A, wherein the center portion of the distal end 147 is further towards the proximal direction than the outer portion of the distal end 147. As such, the distal end 147 may have a shape of a “funnel”.

An interventional device may be introduced through the orifice by moving towards the proximal direction 190 as shown in FIG. 1B.

In one implementation, the thickness of the inner portion 144, the middle portion 143, and the outer portion 142 may be any profile to form a funnel shape. For example but not limited to, the inner portion 144 of the funnel-shaped member 140 may have a thickness smaller than a thickness of the middle portion 143, and the middle portion 143 may have a thickness smaller than a thickness of the outer portion 142. For another example, in one implementation as shown in FIG. 1A, the thickness of the inner portion 144, the middle portion 143, and the outer portion 142 may be gradually decrease from the outer portion towards the inner portion, for example but not limited to, a linear decrease as shown in FIG. 1A.

In one implementation, the funnel-shaped member 140 may be formed from any suitable elastomeric materials, for example but not limited to, silicone, urethane, rubber, polytetrafluoroethylene (PTFE), a polyamide, a polyamide block copolymer, a polyolefin, a polyester, a polyurethane copolymer, polyester copolymers, or blends or copolymers thereof.

In another implementation, the funnel-shaped member 140 may be formed from one or more different materials with different elasticities, for example but not limited to, the outer portion 142 may be stiffer than the inner portion 144, or the inner portion 144 may be more elastic than the outer portion 142. The outer portion 142 may be stiff enough so the funnel-shaped member is fixed inside the housing. The inner portion 144 may be more elastic than the outer portion so that the inner portion may adapt to different shapes and sizes of inserted introducers. In another implementation, a surface of the inner portion may be harder than bulk of the inner portion so as to reduce the likelihood of puncturing the surface of the inner portion by a guide wire.

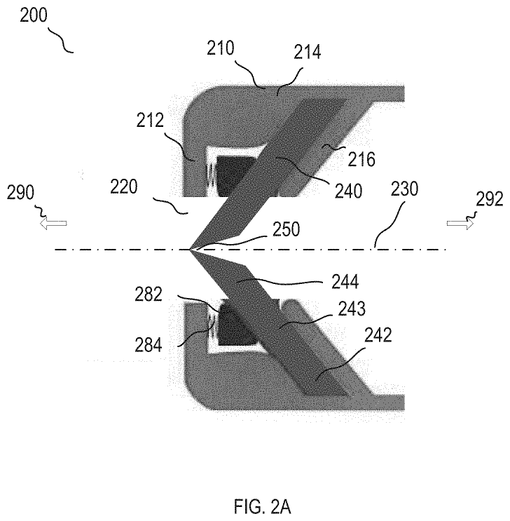

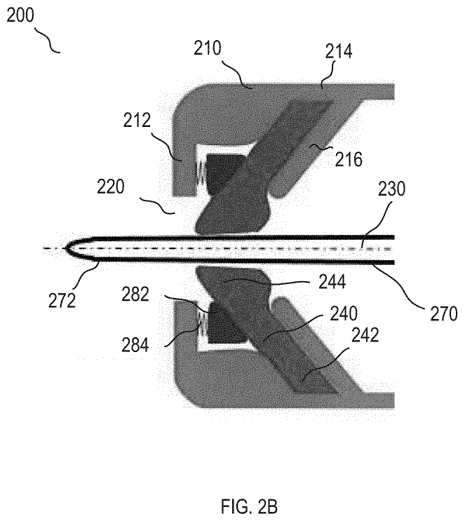

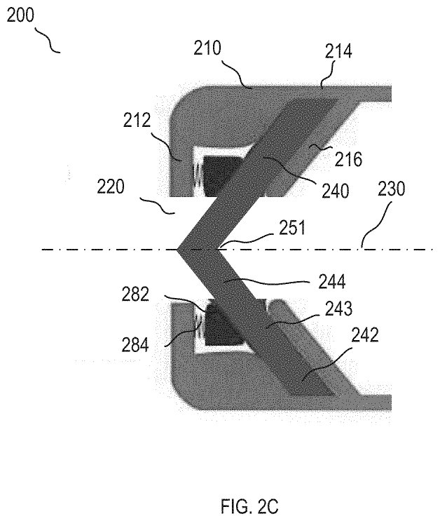

The present disclosure discloses another embodiment of a hemostatic valve 200 with a spring/ring mechanism as shown in FIGS. 2A-2C. The hemostatic valve 200 may include a housing 210 and a funnel-shaped member 240. The housing 210 may have a cylindrical outer shape with a central axis 230. The housing 210 may have a central through portion 220, which may be a through hole and may have the same central axis 230.

The housing 210 may have an outer wall 214, a proximal member 212 disposed at a proximal end of the housing 210, a distal member 216 disposed at a distal direction 292 of the housing 210 relative to the proximal member 212.

The funnel-shaped member 240 may be disposed inside and secured by the housing 210, as shown in FIG. 2A. The funnel-shaped member 240 may include an outer portion 242, a middle portion 243, and an inner portion 244.

The outer portion 242 of the funnel-shaped member 240 may be partially enclosed by the housing 210. In one implementation as shown in FIG. 2A, the outer portion 242 of the funnel-shaped member 240 may have an outer shape similar to the inner shape of enclosure enclosed by the proximal member 212, the outer wall 214, and the distal member 216 of the housing 210, so that the funnel-shaped member 240 may be secured by the housing 210.

The inner portion 244 of the funnel-shaped member 240 may have an elastic orifice 250. The elastic orifice 250 may be at the center of the inner portion 244 and may be aligned with the central axis 230. The inner portion 244 may have a shape as shown in FIG. 2A, so that the elastic orifice 250 has a small thickness along a proximal-distal direction (290 and 292). An outer shape of the inner portion 244 is not limited to the implementation shown in FIG. 2A, for example, the outer shape of the inner portion 244 may have a shape as shown in FIG. 2C.

The hemostatic valve 200 may include the spring/ring mechanism to provide mechanical pressures on the funnel-shaped member 240 to achieve a better and reliable seal. The spring/ring mechanism may optionally include a ring 282 and at least one spring 284.

The spring 284 may be normally under compressed state, so that the spring 284 may provide a pushing force against the ring 282. The hemostatic valve 200 may include a plurality of the spring 284, which are disposed about evenly around the ring 282. For example but not limited to, when a hemostatic valve 200 includes two springs, one spring is disposed about 180 degrees relative to another spring; when a hemostatic valve 200 includes three springs, each spring is disposed about 120 degrees relative to its neighboring spring; and when a hemostatic valve 200 includes four springs, each spring is disposed about 90 degrees relative to its neighboring spring. Here, the term “about” a value may refer to a range of ±10% of the value, inclusive.

The ring 282 may be made from any suitable rigid material to provide pressure on the funnel-shaped member 240, so as to improve seal pressure and achieve a better and reliable seal at the elastic orifice 250.

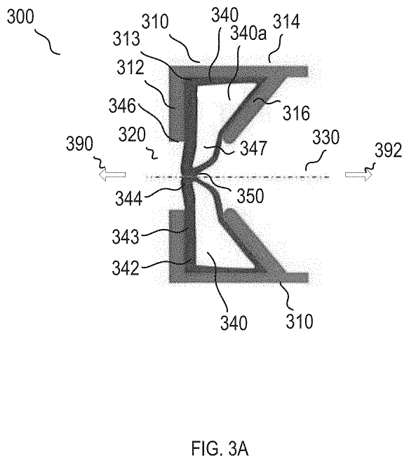

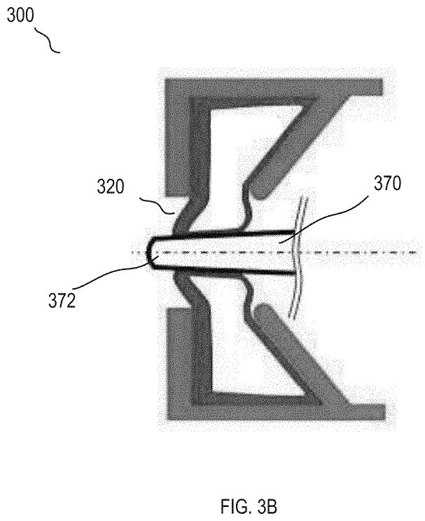

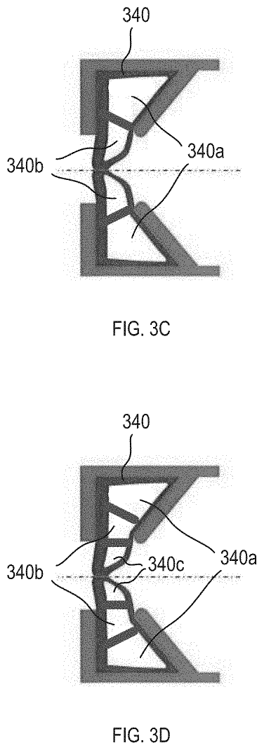

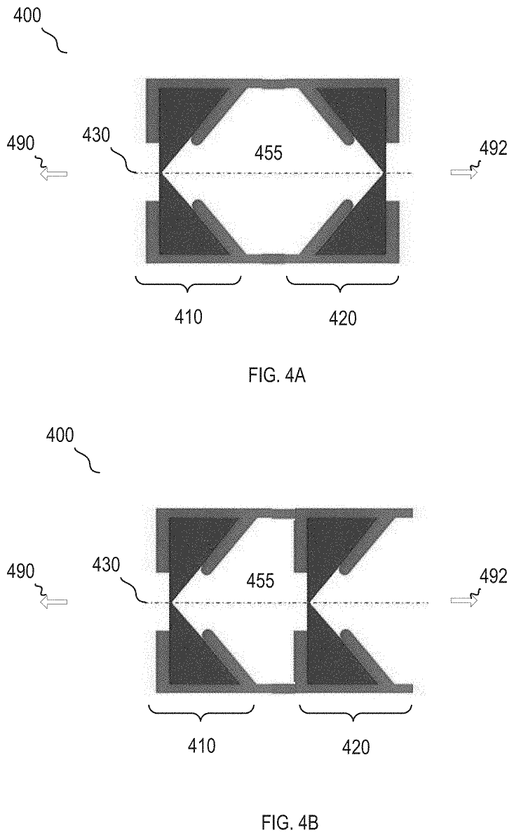

The present disclosure discloses another embodiment of a hemostatic valve 300 as shown in FIGS. 3A and 3B. The hemostatic valve 300 may include a housing 310 and a funnel-shaped member 340. The housing 310 may have a cylindrical outer shape with a central axis 330. The housing 310 may have a central through portion 320, which may be a through hole and may have the same central axis 330.

The funnel-shaped member 340 may be a balloon having a single compartment 340a as shown in FIG. 3A. The compartment 340a may filled with a single type of gas or a mixture of gases (for example, air) with a certain pressure. The gas or one of the gases may be air, Nitrogen gas, Oxygen gas, Carbon Dioxide gas, or noble gas (for example, Helium gas, Neon gas, Argon gas, etc.). The pressure in the compartment 340a may be in a range between 15 pound-force per square inch (PSI) and 100 PSI, inclusive.

The funnel-shaped member 340 may include more than one compartments with same or different gases/pressures. For example, in one implementation as shown in FIG. 3C, the funnel-shaped member 340 may include two compartments: an outer compartment 340a and an inner compartment 340b. The outer compartment 340a and the inner compartment 340b may have different or same gas. The outer compartment may have a lower gas pressure than the inner compartment, so as to improve seal pressure and achieve a better and reliable seal. For example but not limited to, the inner compartment 340b may have a pressure of about 55 PSI, and the outer compartment 340a may have a pressure of about 45 PSI. Here, the term “about” a value may refer to a range of ±10% of the value, inclusive.

In another implementation as shown in FIG. 3D, the funnel-shaped member 340 may include three compartments: an outer compartment 340a, a middle compartment 340b, and an inner compartment 340c. Each of the compartments may have different or same gas, and may have different or same pressure. For example but not limited to, the inner compartment may have a highest gas pressure than the other two compartments, so as to improve seal pressure and achieve a better and reliable seal. For example but not limited to, the inner compartment 340c may have a pressure of about 60 PSI, the middle compartment 340b may have a pressure of about 55 PSI, and the outer compartment 340a may have a pressure of about 45 PSI. Here, the term “about” a value may refer to a range of ±10% of the value, inclusive.

The housing 310 may have an outer wall 314, a proximal member 312 disposed at a proximal end of the housing 310, a distal member 316 disposed at a distal direction 392 relative to the proximal member 312. The outer wall 314, the proximal member 312, and the distal member 316 may define a housing chamber 313. The housing chamber 313 may have a central through portion 320, which may be a through hole and may have a center axis, which is the same as the central axis 330.

The funnel-shaped member 340 may be disposed inside and secured by the housing 310. In one implementation as shown in FIG. 3A, the funnel-shape member 340 may be disposed inside the housing chamber 313. The funnel-shaped member 340 may include an outer portion 342, a middle portion 343, and an inner portion 344.

The outer portion 342 of the funnel-shaped member 340 may be fully or partially enclosed by the housing 310. In one implementation as shown in FIG. 3A, the outer portion 342 of the funnel-shaped member 140 may have an outer shape similar to the inner shape of enclosure enclosed by the proximal member 312, the outer wall 314, and the distal member 316 of the housing 310, so that the funnel-shaped member 340 may be secured by the housing 310.

The inner portion 344 of the funnel-shaped member 340 may have an elastic orifice 350. The elastic orifice 350 may be at the center of the inner portion 344 and may be aligned with the central axis 330.

When no interventional device is introduced as shown in FIG. 3A, the hemostatic valve 300 may be in a closed configuration, the elastic orifice 350 is closed under a combination of an elastic force of the inner portion 344 and internal pressure of the compartment 340a. The closed orifice efficiently provides a seal so that substantially no liquid under a normal hydraulic pressure may pass through the elastic orifice 350. For example but not limited to, no blood under normal human blood pressure may pass through the closed orifice.

When an interventional device 370 is introduced as shown in FIG. 3B, the hemostatic valve 300 may be in an open configuration, and the elastic orifice may be expanded by the interventional device 370 to an open configuration so that a proximal end 372 of the interventional device 370 may pass through. Under the combination of the elastic force of the funnel-shaped member 340 and gas pressure of the compartment 340a, a liquid-tight seal may be created by a contact area between the inner portion 344 of the funnel-shaped member 340 and an outer surface of the interventional device 370. The elastic orifice and the inner portion of the funnel-shaped member may bend along an outer surface of the interventional device so as to increase the contact area between the inner portion of the funnel-shaped member and the interventional device. In this configuration, when the interventional device 370 feeds further towards the proximal direction, the liquid-tight seal between the inner portion 344 of the funnel-shaped member 340 and the outer surface of the interventional device 370 may be maintained. When the interventional device 370 is removed, the orifice may collapse again under the combination of the elastic force of the funnel-shaped member 340 and gas pressure of the compartment 340a so that the hemostatic valve 300 returns to the closed configuration as shown in FIG. 3A again.

In one implementation, the funnel-shaped member 340 may be formed from any suitable elastomeric materials including, for example, silicone, urethane, rubber, polytetrafluoroethylene (PTFE), a polyamide, a polyamide block copolymer, a polyolefin, a polyester, a polyurethane copolymer, polyester copolymers, or blends or copolymers thereof.

In another implementation, the funnel-shaped member 340 may include more than one layers, and each layer may be formed from the suitable elastomeric materials.

In one embodiment wherein the funnel-shaped member 340 includes more than one compartment, each portion of the funnel-shaped member 340 corresponding to each compartment may be made of same material or different materials with different stiffness/elasticity. For one example, the outer compartment 340a may be made of materials with larger stiffness than the inner compartment 340b, or the inner compartment 340b may be more elastic than the outer compartment 340a. For another example, the outer compartment 340a may be made of materials with smaller stiffness than the inner compartment 340b, or the inner compartment 340b may be less elastic than the outer compartment 340a. The outer compartment 340a may be stiff enough so the funnel-shaped member is fixed inside the housing. The inner compartment 340b may be more elastic than the outer compartment so that the inner compartment may adapt to different shapes and sizes of inserted introducers.

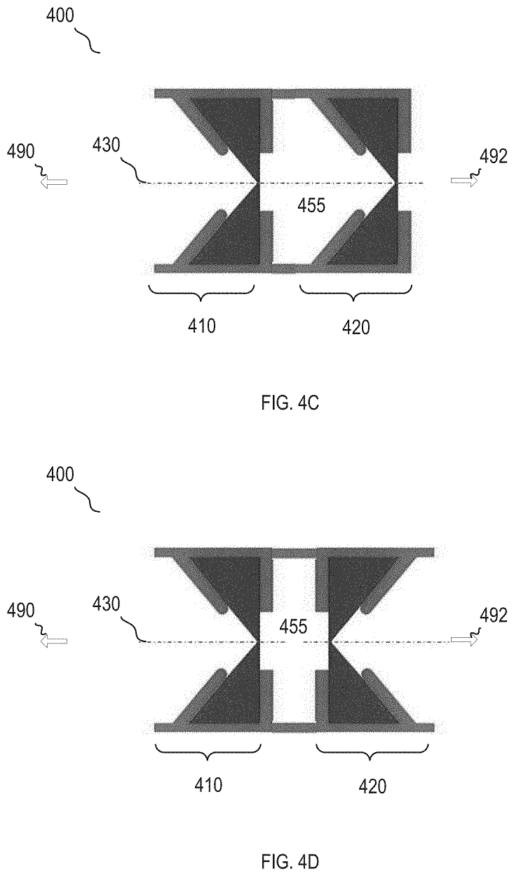

The present disclosure discloses a hemostatic valve system 400 including two or more hemostatic valves in series. As shown in FIGS. 4A-4D, the hemostatic valve system 400 may include a first hemostatic valve 410 and a second hemostatic valve 420 with a same central axis 430. The hemostatic valve system 400 may have a proximal direction 490 and a distal direction 492. The first hemostatic valve 410 and the second hemostatic valve 420 may orient towards the same or different directions, for example, towards the proximal direction 490 or the distal direction 492. The hemostatic valve system including more than one hemostatic valve may provide a better seal when the interventional device inserts or withdraws through the hemostatic valve. The central space 455 between the first and second hemostatic valve may catch and/or trap any stray liquids.

The first hemostatic valve 410 and the second hemostatic valve 420 may be the same or different types of hemostatic valves. For example but not limited to, each of the first hemostatic valve 410 and the second hemostatic valve 420 may be any one of the hemostatic valve 100 in FIGS. 1A-1B, the hemostatic valve 200 in FIGS. 2A-2C, or the hemostatic valve 300 in FIGS. 3A-3D.

For one implementation as shown in FIG. 4A, the first hemostatic valve 410 may be disposed so that a “funnel” shape side of the first hemostatic valve 410 may face towards the distal direction 492; and the second hemostatic valve 420 may be disposed so that a “funnel” shape side of the second hemostatic valve 420 may face towards the proximal direction 490.

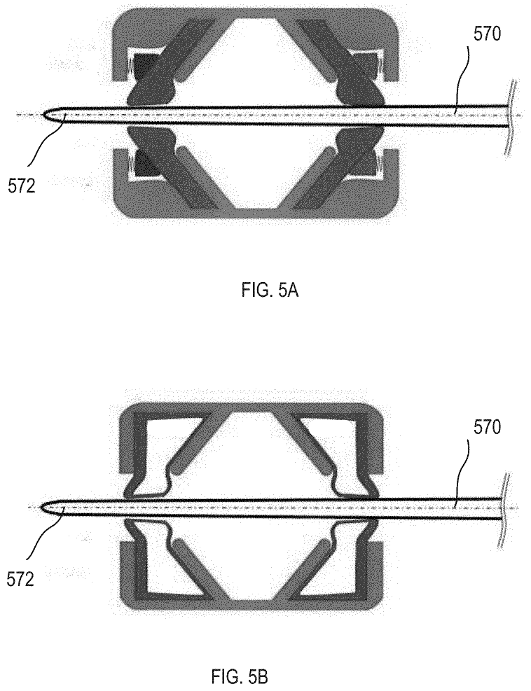

FIG. 5A shows one example wherein both hemostatic valve 410 and 420 are the hemostatic valve 200 in FIGS. 2A-2B and a proximal end 572 of an interventional device 570 may pass through the hemostatic valve system. FIG. 5B shows another example wherein both hemostatic valve 410 and 420 are the hemostatic valve 300 in FIGS. 3A-3B and a proximal end 572 of an interventional device 570 may pass through the hemostatic valve system.

For another implementation as shown in FIG. 4B, the first hemostatic valve 410 may be disposed so that the “funnel” side of the first hemostatic valve 410 may face towards the distal direction 492; and the second hemostatic valve 420 may be disposed so that the “funnel” side of the second hemostatic valve 420 may face towards the distal direction 492.

For another implementation as shown in FIG. 4C, the first hemostatic valve 410 may be disposed so that the “funnel” side of the first hemostatic valve 410 may face towards the proximal direction 490; and the second hemostatic valve 420 may be disposed so that the “funnel” side of the second hemostatic valve 420 may face towards the proximal direction 490.

For another implementation as shown in FIG. 4D, the first hemostatic valve 410 may be disposed so that the “funnel” side of the first hemostatic valve 410 may face towards the proximal direction 490; and the second hemostatic valve 420 may be disposed so that the “funnel” side of the second hemostatic valve 420 may face towards the distal direction 492.

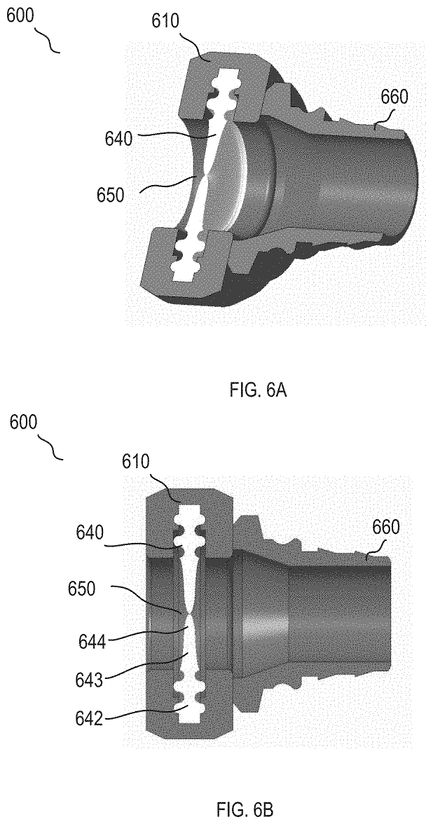

The present disclosure describes an embodiment of another hemostatic valve 600 as shown in FIGS. 6A-6B. One or more hemostatic valves 600 or in combination with other types of hemostatic valves may be used to form a hemostatic valve system as discussed above.

The hemostatic valve 600 may include a housing 610, a disk 640 having a central hole with circumferential uniform seal 650. Optically, the hemostatic valve 600 may include a sheath coupling portion 660 adapted to couple and/or connect to a sheath.

The disk 640 may be made of elastic materials and include an outer portion 642, a middle portion 643, and an inner portion 644. The outer portion 642 may be made of materials with larger stiffness so as to be fixed in the housing. The outer portion 642 may include expansion grooves to allow disk material compression and/or to slide along an arrow direction 645 upon a large introducer being inserted. The middle portion 643 may have a tapered region to provide strength for large introducers. The inner portion 644 may have an S-shaped surface to support self-centering guidance of an introducer and be made from a bendable self-back-flipping membrane.

While the particular invention has been described with reference to illustrative embodiments, this description is not meant to be limiting. Various modifications of the illustrative embodiments and additional embodiments of the invention will be apparent to one of ordinary skill in the art from this description. Those skilled in the art will readily recognize that these and various other modifications can be made to the exemplary embodiments, illustrated and described herein, without departing from the spirit and scope of the present invention. It is therefore contemplated that the appended claims will cover any such modifications and alternate embodiments. Certain proportions within the illustrations may be exaggerated, while other proportions may be minimized. Accordingly, the disclosure and the figures are to be regarded as illustrative rather than restrictive.

1. A system for maintaining hemostasis during introducing or withdrawing an interventional device, the system comprising:
a housing comprising a first end, a second end and a side wall defining a housing chamber between the first end and the second end, the housing comprising a through channel disposed along a central axis of the housing;
a first funnel-shaped member disposed at least partially within the housing chamber, wherein:
the first funnel-shaped member comprises an inner portion, a middle portion and an outer portion, and
the inner portion of the first funnel-shaped member comprises an elastic orifice, the elastic orifice being aligned with the through channel of the housing; and
the first funnel-shaped member comprising an open configuration and a closed configuration, wherein:
when the first funnel-shaped member is in the closed configuration, the elastic orifice is closed, and
when the first funnel-shaped member is in the open configuration, an interventional device is introduced through the system and the elastic orifice is open.

2. The system according to claim 1, wherein the first funnel-shaped member is made from at least one elastic material.

3. The system according to claim 1, wherein:
the first funnel-shaped member comprises a first end and a second end; and
when the first funnel-shaped member is in the closed configuration, the second end of the first funnel-shaped member is funnel-shaped.

4. The system according to claim 3, wherein when the first funnel-shaped member is in the open configuration, the interventional device is introduced from the second end of the first funnel-shaped member towards the first end of the first funnel-shaped member.

5. The system according to claim 3, further comprising:
a ring and a set of springs disposed between the first end of the first funnel-shaped member and the first end of the housing.

6. The system according to claim 1, wherein the first funnel-shaped member comprises one or more compartments and each of the one or more compartments is filled with a gas at a certain gas pressure.

7. The system according to claim 6, wherein:
the gas comprises at least one of air, Nitrogen gas, Oxygen gas, Carbon Dioxide gas, or noble gases; and
the certain gas pressure comprises a range of between 15 pound-force per square inch (PSI) and 100 PSI.

8. The system according to claim 1, wherein the first funnel-shaped member comprises an outer compartment and an inner compartment, the outer compartment is further away from the central axis than the inner compartment.

9. The system according to claim 8, wherein:
the outer compartment comprises a first gas at a first gas pressure; and
the inner compartment comprises a second gas at a second gas pressure, wherein the first gas pressure is smaller than the second gas pressure.

10. The system according to claim 9, wherein the first gas pressure is about 45 PSI and the second gas pressure is about 55 PSI.

11. A system for maintaining hemostasis during introducing or withdrawing an interventional device, the system comprising:
a housing comprising a first end, a second end and a side wall defining a housing chamber between the first end and the second end, the housing comprising a through channel disposed along a central axis of the housing;
a first funnel-shaped member disposed at least partially within the housing chamber, wherein:
the first funnel-shaped member comprises an inner portion, a middle portion and an outer portion,
the inner portion of the first funnel-shaped member comprises an elastic orifice, the elastic orifice being aligned with the through channel of the housing, and
the first funnel-shaped member comprising an open configuration and a closed configuration, wherein when the first funnel-shaped member is in the closed configuration, the elastic orifice is closed, and when the first funnel-shaped member is in the open configuration, an interventional device is introduced through the system and the elastic orifice is open;
a second funnel-shaped member disposed at least partially within the housing chamber, wherein:
the second funnel-shaped member comprises an inner portion, a middle portion and an outer portion,
the inner portion of the second funnel-shaped member comprises an elastic orifice, the elastic orifice being aligned with the through channel of the housing, and
the second funnel-shaped member comprising an open configuration and a closed configuration, wherein when the second funnel-shaped member is in the closed configuration, the elastic orifice is closed, and when the second funnel-shaped member is in the open configuration, the interventional device is introduced through the system and the elastic orifice is open.

12. The system according to claim 11, wherein:
the first funnel-shaped member comprises a first end and a second end;
when the first funnel-shaped member is in the closed configuration, the second end of the first funnel-shaped member is funnel-shaped;
the second funnel-shaped member comprises a first end and a second end; and
when the second funnel-shaped member is in the closed configuration, the second end of the second funnel-shaped member is funnel-shaped.

13. The system according to claim 12, wherein:
the first funnel-shaped member is made of at least one of elastic materials; and
the second funnel-shaped member is made of at least one of elastic materials.

14. The system according to claim 12, wherein:
the first funnel-shaped member comprises one or more compartments and each of the one or more compartments is filled with a gas at a certain gas pressure; and
the second funnel-shaped member comprises one or more compartments and each of the one or more compartments is filled with a gas at a certain gas pressure.

15. The system according to claim 12, wherein:
the first funnel-shaped member comprises one or more compartments and each of the one or more compartments is filled with a gas at a certain gas pressure; and
the second funnel-shaped member is made of at least one of elastic materials.

16. The system according to claim 12, wherein:
the first end of the first funnel-shaped member faces towards a proximal direction of the system and the first end of the second funnel-shaped member faces towards a distal direction of the system; and
when the first funnel-shaped member and the second funnel-shaped member are in the open configuration, the interventional device is introduced from the first end of the second funnel-shaped member towards the second end of the second funnel-shaped member, and from the second end of the first funnel-shaped member towards the first end of the first funnel-shaped member.

17. The system according to claim 12, wherein:
the second end of the first funnel-shaped member faces towards a proximal direction of the system and the second end of the second funnel-shaped member faces towards a distal direction of the system; and
when the first funnel-shaped member and the second funnel-shaped member are in the open configuration, the interventional device is introduced from the second end of the second funnel-shaped member towards the first end of the second funnel-shaped member, and from the first end of the first funnel-shaped member towards the second end of the first funnel-shaped member.

18. The system according to claim 12, wherein:
the first end of the first funnel-shaped member faces towards a proximal direction of the system and the second end of the second funnel-shaped member faces towards a distal direction of the system; and
when the first funnel-shaped member and the second funnel-shaped member are in the open configuration, the interventional device is introduced from the second end of the second funnel-shaped member towards the first end of the second funnel-shaped member, and from the second end of the first funnel-shaped member towards the first end of the first funnel-shaped member.

19. The system according to claim 12, wherein:
the second end of the first funnel-shaped member faces towards a proximal direction of the system and the first end of the second funnel-shaped member faces towards a distal direction of the system; and
when the first funnel-shaped member and the second funnel-shaped member are in the open configuration, the interventional device is introduced from the first end of the second funnel-shaped member towards the second end of the second funnel-shaped member, and from the first end of the first funnel-shaped member towards the second end of the first funnel-shaped member.

20. A method for maintaining hemostasis during introducing or withdrawing an interventional device, the method comprising:
introducing or withdrawing an interventional device through a hemostatic valve system, the hemostatic valve system comprising:
a housing comprising a first end, a second end and a side wall defining a housing chamber between the first end and the second end, the housing comprising a through channel disposed along a central axis of the housing;
a first funnel-shaped member disposed at least partially within the housing chamber, wherein:
the first funnel-shaped member comprises an inner portion, a middle portion and an outer portion, and
the inner portion of the first funnel-shaped member comprises an elastic orifice, the elastic orifice being aligned with the through channel of the housing; and
the first funnel-shaped member comprising an open configuration and a closed configuration, wherein:
when the first funnel-shaped member is in the closed configuration, the elastic orifice is closed, and
when the first funnel-shaped member is in the open configuration, the interventional device is introduced through the hemostatic valve system and the elastic orifice is open.
